(19) United States
(12) Patent Application Publication
(10) Pub. No.: US 2006/0009698 A1
(43) Pub. Date: Jan. 12, 2006

(54) HAND-HELD MONITOR FOR MEASURING VITAL SIGNS

(75) Inventors: Matthew John Banet, Del Mar, CA (US); Zhou Zhou, La Jolla, CA (US)

Correspondence Address: MATTHEW J. BANET, 6540 LUSK BLVD., C200, SAN DIEGO, CA, 92121, US

(73) Assignee: TRIAGE WIRELESS, INC., San Diego, CA (US)

(21) Appl. No.: 11/162,742

(22) Filed: Sep. 21, 2005

Related U.S. Application Data
(63) Continuation-in-part of application No. 10/709,014, filed on Apr. 7, 2004.

(51) Int. Cl. A61B 5/02

(52) U.S. Cl. 600/485; 128/903

(57) ABSTRACT

The invention provides a monitor for measuring blood pressure and other vital signs from a patient without using a cuff. The monitor features a housing with a first surface that, in turn, supports a first sensor. The first sensor features: i) an optical system with one or more light sources (e.g., LEDs or laser diodes) that generate optical radiation, and a photodetector oriented to collect radiation after it irradiates the patient and in response generate an optical signal; and ii) a first electrode. A second sensor features a second electrode paired with the first electrode that collects an electrical signal from the patient. A microprocessor in electrical communication with the first and second sensor receives the optical and electrical signals and processes them with an algorithm to determine systolic and diastolic blood pressure.

### BACKGROUND OF THE INVENTION

The present invention relates to medical devices for monitoring vital signs such as heart rate, pulse oximetry, and blood pressure.

### DESCRIPTION OF THE RELATED ART

Pulse oximeters are medical devices featuring an optical module, typically worn on a patient's finger or ear lobe, and a processing module that analyzes data generated by the optical module. The optical module typically includes first and second light sources (e.g., light-emitting diodes, or LEDs) that transmit optical radiation at, respectively, red (λ˜630-670 nm) and infrared (λ˜800-1200 nm) wavelengths. The optical module also features a photodetector that detects radiation transmitted or reflected by an underlying artery. Typically the red and infrared LEDs sequentially emit radiation that is partially absorbed by blood flowing in the artery. The photodetector is synchronized with the LEDs to detect transmitted or reflected radiation. In response, the photodetector generates a separate radiation-induced signal for each wavelength. The signal, called a plethysmograph, varies in a time-dependent manner as each heartbeat varies the volume of arterial blood and hence the amount of transmitted or reflected radiation. A microprocessor in the pulse oximeter processes the relative absorption of red and infrared radiation to determine the oxygen saturation in the patient's blood. A number between 94%-100% is considered normal, while a value below 85% typically indicates the patient requires hospitalization. In addition, the microprocessor analyzes time-dependent features in the plethysmograph to determine the patient's heart rate.

Another medical device, called a sphygmomanometer, measures a patient's blood pressure using an inflatable cuff and a sensor (e.g., a stethoscope) that detects blood flow by listening for sounds called the Korotkoff sounds. During a measurement, a medical professional typically places the cuff around the patient's arm and inflates it to a pressure that exceeds the systolic blood pressure. The medical professional then incrementally reduces pressure in the cuff while listening for flowing blood with the stethoscope. The pressure value at which blood first begins to flow past the deflating cuff, indicated by a Korotkoff sound, is the systolic pressure. The stethoscope monitors this pressure by detecting strong, periodic acoustic ‘beats’ or ‘taps’ indicating that the blood is flowing past the cuff (i.e., the systolic pressure barely exceeds the cuff pressure). The minimum pressure in the cuff that restricts blood flow, as detected by the stethoscope, is the diastolic pressure. The stethoscope monitors this pressure by detecting another Korotkoff sound, in this case a ‘leveling off’ or disappearance in the acoustic magnitude of the periodic beats, indicating that the cuff no longer restricts blood flow (i.e., the diastolic pressure barely exceeds the cuff pressure).

Low-cost, automated devices measure blood pressure using an inflatable cuff and an automated acoustic or pressure sensor that measures blood flow. These devices typically feature cuffs fitted to measure blood pressure in a patient's wrist, arm or finger. During a measurement, the cuff automatically inflates and then incrementally deflates while the automated sensor monitors blood flow. A microcontroller in the automated device then calculates blood pressure. Cuff-based blood-pressure measurements such as these typically only determine the systolic and diastolic blood pressures; they do not measure dynamic, time-dependent blood pressure.

Data indicating blood pressure are most accurately measured during a patient's appointment with a medical professional, such as a doctor or a nurse. Once measured, the medical professional can manually record these data in either a written or electronic file. Appointments typically take place a few times each year. Unfortunately, in some cases, patients experience ‘white coat syndrome’ where anxiety during the appointment affects the blood pressure that is measured. For example, white coat syndrome can elevate a patient's heart rate and blood pressure; this, in turn, can lead to an inaccurate diagnoses.

### SUMMARY OF THE INVENTION

In one aspect, the invention provides a monitor for measuring blood pressure and other vital signs from a patient without using a cuff. The monitor features a housing with a first surface that, in turn, supports a first sensor. The first sensor features: i) an optical system with one or more light sources (e.g., LEDs or laser diodes) that generate optical radiation, and a photodetector oriented to collect radiation after it irradiates the patient and in response generate an optical signal; and ii) a first electrode. A second sensor features a second electrode paired with the first electrode that collects an electrical signal from the patient. A microprocessor in electrical communication with the first and second sensor receives the optical and electrical signals and processes them with an algorithm to determine systolic and diastolic blood pressure, as is described in more detail below.

In embodiments, both the first and second sensors are disposed on the first surface, e.g. the monitor's front surface. In this case, for example, the patient makes a measurement by holding the monitor so that a finger from one hand contacts the first sensor, and a finger from the other hand contacts the second sensor. In another embodiment, the housing features a surface (e.g., a back surface) that supports the first sensor. In this case, the patient holds the monitor so a finger from one hand contacts the first sensor on the back surface. The patient then initiates a measurement through a user interface on the front surface, and after a short delay presses a finger from the other hand against the second sensor on the front surface to complete the measurement.

In typical embodiments the first surface houses both the light-emitting diode and photodetector. In this case, the first surface can include the first electrode, typically shaped as an annular ring, which surrounds both the light source and the photodetector. Alternatively, the first electrode can be a metal pad disposed adjacent to the light source and photodetector.

In one embodiment the first surface features a depression that comprises the first sensor. The depression typically has a width that is approximately the size of a human finger (e.g., between 1 and 2 cm). In this case, first surface is the monitor's back surface, and the patient holds the device and gently slides their finger into the depression on the back surface. Both the light source and photodiode may be disposed in the depression, in which case the first sensor operates in a ‘reflection mode’ geometry wherein radiation from the light source irradiates an artery in the patient's finger. A portion of the radiation reflects off the artery and is modulated by a volumetric change in the artery brought on by blood pulsing therein. The photodetector detects the reflected portion of the radiation, and in response generates an optical signal that after further processing is transformed to the optical waveform. In a related embodiment, the monitor includes a component, such as a plastic piece or a flexible strap, which attaches to the first surface to partially cover the first sensor so that the optical system operates in a ‘transmission mode’ geometry. The component typically includes the first light source and orients it so that radiation passes through the patient's fingernail and underlying artery. The photodetector detects a portion of the transmitted radiation modulated by flowing blood and in response generates an optical signal.

The invention features many advantages, the most notable being it provides a small-scale, hand-held monitor that measures blood pressure and other vital signs without using a cuff. This results in a comfortable measurement that the patient can easily perform throughout the day. Once the information is collected, the monitor can store it in a computer memory, and then transmit it through a Universal Serial Bus (USB) or through wireless means to an Internet-based computer system. In this way, both the patient and an associated medical professional can view the information from a website, and in response can characterize a patient's real-time vital signs during their day-to-day activities, rather than rely on an isolated measurement during a medical check-up. For example, by viewing this information, a physician can delineate between patients exhibiting white coat hypertension and patients who truly have high blood pressure. In response, the physician can prescribe medication and then monitor how this affects the patient's blood pressure. These and other advantages are described in detail in the following description, and in the claims.

### BRIEF DESCRIPTION OF THE DRAWINGS

FIG. 1A is a semi-schematic view of a portable, hand-held vital sign monitor featuring first and second pad sensors that measure blood pressure, pulse oximetry, and heart rate;

FIG. 1B is a semi-schematic view of the vital sign monitor of FIG. 1A in use by a patient;

FIG. 2 is a graph of time-dependent optical and electrical waveforms generated by the first and second pad sensors of FIG. 1A;

FIGS. 3A and 3B are semi-schematic top views of the first and second pad sensors used in the vital sign monitor of FIG. 1A.

FIG. 4 is a schematic diagram of the electrical components of the vital sign monitor of FIG. 1A;

FIG. 5 is a semi-schematic view of the monitor of FIG. 1A connecting through a USB port to either a personal computer or a wireless device;

FIGS. 6A and 6B are schematic views of an Internet-based system that receives information from the vital sign monitor of FIG. 1A through, respectively, a wired or wireless connection;

FIGS. 7A and 7B are, respectively, front and back views of a vital sign monitor according to an alternate embodiment of the invention featuring a pad sensor operating in a ‘reflection’ mode; and

FIG. 8 is a back view of a vital sign monitor operating in a ‘transmission’ mode.

### DETAILED DESCRIPTION OF THE INVENTION

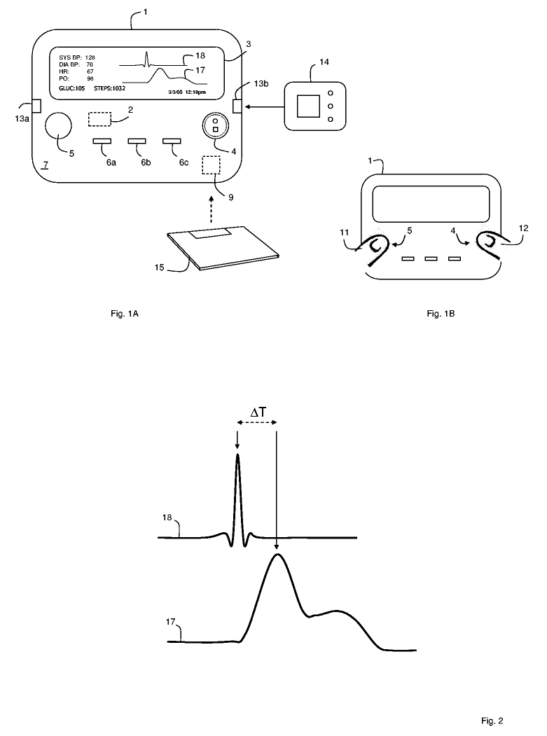

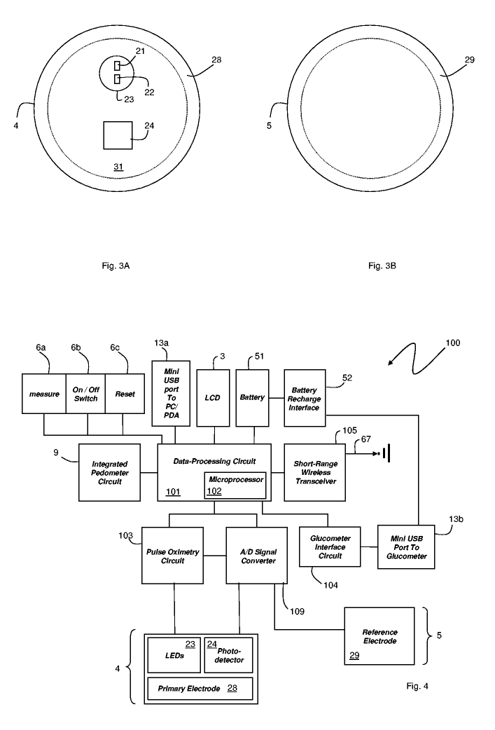

FIGS. 1A and 1B show a portable, hand-held vital sign monitor 1 that measures a patient's systolic and diastolic blood pressure, pulse oximetry, and heart rate using a pair of ‘pad’ sensors 4, 5 mounted on the monitor's top surface 7. As shown in more detail in FIGS. 3A, 3B, the second pad sensor 4 features an optical source 23 that includes red 21 and infrared 23 LEDs that irradiate a patient's finger, and a photodetector 24 that detects reflected radiation to generate a time-resolved optical signal 17. The second pad sensor 4 also includes an electrode 28 shaped as an annular ring that, when coupled with a reference electrode 29 in the first pad sensor 5, generates a time-resolved electrical signal 18. An algorithm (described in detail below) operating on a microprocessor within the vital sign monitor 1 processes the time-resolved optical 17 and electrical 18 signals to determine the patient's blood pressure, heart rate, and pulse oximetry.

In addition to these properties, the vital sign monitor 1 includes an integrated pedometer circuit 9 that measures steps and, using an algorithm, calories burned. To receive information from external devices, the monitor 1 also includes: i) Universal Serial Bus (USB) connectors 13a, 13b that connect and download information from an external glucometer 14 and other devices with serial interfaces; and ii) a short-range wireless transceiver 2 that receives information such as body weight and percentage of body fat from an external scale 15. The patient views information from a liquid crystal display (LCD) display 3, and can interact with the monitor 1 (e.g., reset or reprogram it) using a series of buttons 6a, 6b, 6c.

Referring to FIG. 1B, during use the patient contacts the first pad sensor 5 with a finger 11 from their left hand, and the second pad sensor 4 with a finger 12 from their right hand. The patient's fingers 11, 12 stay in contact with the first 5 and second 4 pad sensors until these sensors measure the optical 17 and electrical 18 waveforms with a suitable signal-to-noise ratio; this typically takes a few seconds. Each waveform features a separate ‘pulse’, generated with each heartbeat, which the algorithm collectively processes to determine the patient's vital signs. Specifically, following each heartbeat, an electrical impulse travels essentially instantaneously from the patient's heart to the fingers 11, 12, where the electrodes detect it to generate the electrical waveform 18. At a later time, a pressure wave induced by the same heartbeat propagates through the patient's arteries and arrives at an artery in the right finger 12, where the LEDs and photodetector in the second pad sensor detect it by optically measuring a time-dependent volumetric change in the artery to generate the optical waveform 17. The propagation time of the electrical impulse is independent of blood pressure, whereas the propagation time of the pressure wave depends strongly on pressure, as well as mechanical properties of the patient's arteries (e.g., arterial size, stiffness). The algorithm analyzes the time difference ΔT between the arrivals of these signals, i.e. the relative occurrence of the optical 17 and electrical 18 waveforms as measured by the first and second pad sensors. Calibrating the measurement (e.g., with a conventional blood pressure cuff at an earlier time) accounts for patient-to-patient variations in arterial properties, and correlates ΔT to both systolic and diastolic blood pressure. This results in a calibration table. During an actual measurement, the calibration source is removed, and the microprocessor analyzes ΔT along with other properties of the optical and electrical waveforms and the calibration table to calculate the patient's real-time blood pressure.

The microprocessor can analyze other properties of the optical waveform 17 to augment the above-mentioned measurement of blood pressure. For example, the waveform can be ‘fit’ using a mathematical function that accurately describes the waveform's features, and an algorithm (e.g., the Marquardt-Levenberg algorithm) that iteratively varies the parameters of the function until it best matches the time-dependent features of the waveform. In this way, blood pressure-dependent properties of the waveform, such as its width, rise time, fall time, and area, can be calibrated as described above.

Methods for processing optical and electrical waveforms to determine blood pressure without using a cuff are described in the following co-pending patent applications, the entire contents of which are incorporated by reference: 1) CUFFLESS BLOOD-PRESSURE MONITOR AND ACCOMPANYING WIRELESS, INTERNET-BASED SYSTEM (U.S. Ser. No. 10/709,015; filed Apr. 7, 2004); 2) CUFFLESS SYSTEM FOR MEASURING BLOOD PRESSURE (U.S. Ser. No. 10/709,014; filed Apr. 7, 2004); 3) CUFFLESS BLOOD PRESSURE MONITOR AND ACCOMPANYING WEB SERVICES INTERFACE (U.S. Ser. No. 10/810,237; filed Mar. 26, 2004); 4) VITAL SIGN MONITOR FOR ATHLETIC APPLICATIONS (U.S. Ser. No. ______; filed Sep. 13, 2004); 5) CUFFLESS BLOOD PRESSURE MONITOR AND ACCOMPANYING WIRELESS MOBILE DEVICE (U.S. Ser. No. 10/967,511; filed Oct. 18, 2004); and 6) BLOOD PRESSURE MONITORING DEVICE FEATURING A CALIBRATION-BASED ANALYSIS (U.S. Ser. No. 10/967,610; filed Oct. 18, 2004); 7) PERSONAL COMPUTER-BASED VITAL SIGN MONITOR (U.S. Ser. No. 10/906,342; filed Feb. 15, 2005); 8) PATCH SENSOR FOR MEASURING BLOOD PRESSURE WITHOUT A CUFF (U.S. Ser. No. 10/906,315; filed Feb. 14, 2005); 9) SMALL-SCALE, VITAL-SIGNS MONITORING DEVICE, SYSTEM AND METHOD (U.S. Ser. No. 10/907,440; filed Mar. 31, 2005); and 10) PATCH SENSOR SYSTEM FOR MEASURING VITAL SIGNS (U.S. Ser. No. 11/160957; filed Jul. 18, 2005);

FIG. 4 shows a schematic drawing of the collective electronic components 100 within the monitor described in FIGS. 1A and 1B. A data-processing circuit 101 features a microprocessor 102 that operates the above-described algorithm for calculating blood pressure from the optical and electrical waveforms. The data-processing circuit 101 also controls a pulse oximetry circuit 103 that connects to the LEDs 23 within the second pad sensor 4, and an analog-to-digital converter 109 that receives an analog signal from the photodetector 24 also within the second pad sensor. During operation, the pulse oximetry circuit 103 powers a first LED (e.g., an LED emitting a red wavelength), which in response emits radiation. As the heart pumps blood through an artery in the patient's finger, blood cells absorb and transmit varying amounts of the radiation depending on how much oxygen binds to the cells' hemoglobin. A portion of the radiation reflects off the artery into the photodetector 24, which in response registers a photocurrent. The photocurrent converts into a voltage that is digitized by the analog-to-digital converter 109 and sent to the pulse oximetry circuit 103 for processing. This process is repeated at a later time (e.g., a few milliseconds later) for a second LED (e.g., an LED emitting an infrared wavelength), and then continually repeated until the pulse oximetry circuit 103 collects a time-resolved optical waveform for each wavelength. The pulse oximetry circuit 103 then sends the optical waveforms to the data-processing circuit 101, where the microprocessor 102 processes them to determine blood pressure as described above, as well as heart rate and pulse oximetry using algorithms known in the art.

Concurrent with measurement of the optical waveform, the primary electrode 28 in the first pad sensor 4 and the reference electrode 29 in the second pad sensor 5 detect an electrical impulse generated in the patient following a heartbeat. This signal, which is similar to that collected with a conventional 2-lead ECG system, is registered as an analog voltage which is then digitized by the analog-to-digital converter 109 to form the electrical waveform. The data-processing circuit 101 receives the electrical waveform, and sends it to the microprocessor 102 for processing with the optical waveform.

During operation, an LCD 3 displays both the optical and electrical waveforms, as well as any vital signs determined from these waveforms. To collect other data inputs, the monitor also includes a glucometer interface circuit 104 that connects to an external glucometer through a mini USB port 13b to collected glucose information, and an integrated pedometer circuit 9 that measures steps and, along with a heart rate value, calories burned. All information can be sent from the monitor to an external device, such as a personal computer 130 or PDA 72 as described with reference to FIGS. 6A and 6B, using a short-range wireless transceiver 105 and an antenna 67. The monitor can also send information to these devices using a conventional USB port 13a.

The short-range wireless transceiver 105 can also receive information, such as weight and body-fat percentage, from an external scale. A battery 51 powers all the electrical components within the monitor, and is preferably a metal hydride battery (generating 3-7V) that can be recharged through a battery-recharge interface 52. The battery-recharge interface 52 can receive power through a serial port, e.g. a computer's USB port 13b. Buttons control functions within the monitor such as a manual measurement switch 6a, on/off switch 6b, and a system reset 6c.

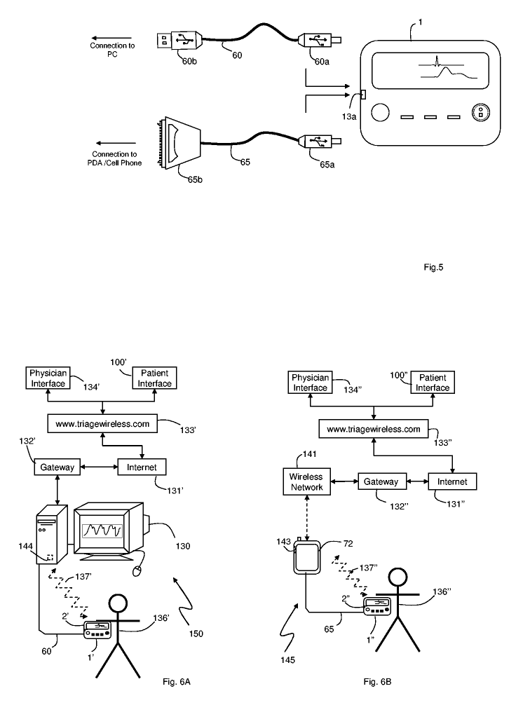

Referring to FIG. 5, to transfer information to Internet-accessible devices, the monitor 1 includes a mini USB port 13a that connects to a personal computer through a conventional USB connector 60b terminating a first cable 60. Alternatively, the monitor connects to a personal digital assistant (PDA) through a serial connector 65b terminating a second cable 65. The PDA, for example, can be a conventional wireless device, such as a cellular phone.

FIGS. 6A and 6B show preferred embodiments of Internet-based systems 150, 145 that operate in concert with the vital sign monitor 1′, 1″ to send information from the patient 136′, 136″ to an Internet-accessible website 133′, 133″. There, a user can access the information using a conventional web browser through a ‘patient’ interface 100′, 100″ or a ‘physician’ interface 134′, 134″. Typically the patient interface 100′, 100″ shows information from a single user, whereas the physician interface 134′, 134″ displays information for multiple patients. In both cases, information flows from the monitor 1′, 1 through a USB cable 60, 65 to an external device (e.g., a personal computer 130 or PDA 72). The personal computer 130 connects to the Internet 131′ through a wired gateway software system 132′, such as an Internet Service Provider. Alternatively, the monitor 1″ wirelessly sends information through a wireless network 141 to a wireless gateway 132″, which then transfers the information to the Internet 131″.

In other embodiments, the multifunctional vital sign monitor 1′, 1″ transmits patient information using a short-range wireless transceiver 2′, 2″ through a short-range wireless connection 137′, 137″ (e.g., Bluetooth, 802.15.4, part-15) to either the personal computer 130 or PDA 72. For example, the vital sign monitor 1′,1″ can transmit to a matched transceiver 144 within (or connected to) the personal computer 130, or alternatively to a transceiver 143 within the PDA 72. In both cases, the monitor collects and stores information from the patient 136′, 136″, and then transmits this when it roams within range of the personal computer 130 or PDA 72.

During typical operation, the patient 136′,136″ uses the monitor 1′ 1″ for a period of time ranging from several hours to several months. Typically the patient 136′,136″ takes measurements a few times throughout the day, and then uploads the information to the Internet-based systems 150, 145 using a wired or wireless connection. To view patient information sent from the monitor 1′,1″, the patient 136′, 136″ (or other user) accesses the appropriate user interface hosted on the website 133′, 133″ through the Internet 131′,131″.

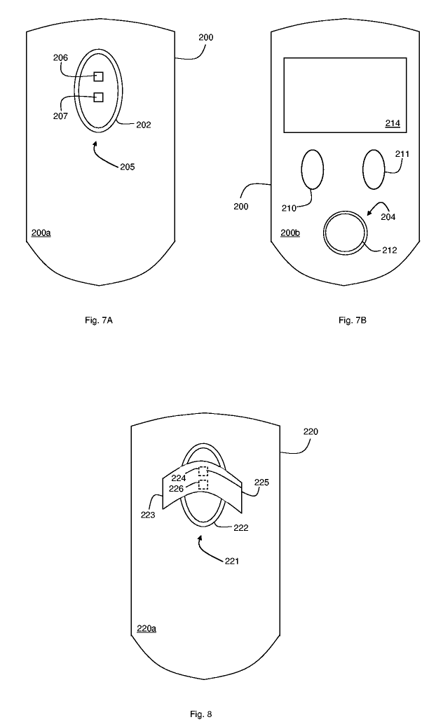

FIGS. 7A and 7B shows another embodiment of the hand-held vital sign monitor 200 that includes a first pad sensor 205 on the monitor's back surface 200a, and a second pad sensor 204 on the monitor's front surface 200b. The monitor 200 is designed to measure vital signs and other properties while easily fitting in the palm of a patient's hand. Similar to the monitor shown in FIG. 1A, the first pad sensor 205 includes a pair of LEDs 206 and a photodetector 207, along with a primary electrode 202. The second pad sensor 204 features a reference electrode 212. Working in concert, the first 205 and second 204 pad sensors measure optical and electrical waveforms as described above. During operation, a patient holds the monitor 200 in the palm of one hand and places a finger on the first pad sensor 205. The patient then initiates a measurement with a pair of buttons 210, 211 on the monitor's front surface 200b. Using an LCD 214, the monitor 200 prompts the patient to keep the finger from one hand on the first pad sensor 205, and then place a finger from the other hand on the second pad sensor 204. The monitor then makes an optical measurement in a ‘reflection mode’ to measure an optical waveform, and an electrical measurement to measure an electrical waveform, as described above. This information is then processed to determine the patient's vital signs.

FIG. 8 shows an embodiment of the invention related to that shown in FIGS. 7A and 7B where the monitor 220 has a back surface 220a that replaces the back surface shown in FIG. 7A. In this case, the back surface 220a features a first pad sensor 221 that makes an optical measurement in a ‘transmission mode’. The first pad sensor 221 includes a flexible strap 223 that houses a pair of LEDs 224 that emit red and infrared radiation. A photodiode 226 embedded in the back surface 220a detects radiation that transmits through arteries within the patient's finger. An electronic ribbon cable 225 featuring electrical leads connects the pair of LEDs 224 and the photodiode 226 to electronics (similar to that shown in FIG. 4) in the monitor. During a measurement, the patient slips a finger in the pad sensor and underneath the flexible strap 223 so that radiation emitted by the LEDs 224 passes through the finger and into the photodiode 226 to generate the optical waveform. A primary electrode 222 measures the electrical waveform in combination with a second pad sensor similar to that shown in FIG. 7B. Unlike an optical system with a ‘reflection mode’ geometry, such as that shown in FIGS. 7A and 7B, the optical system with the ‘transmission mode’ geometry shown in FIG. 8 detects relatively small amounts of background radiation, i.e. stray radiation directly from the LEDs or reflected off a surface of the patient's finger. This results in a high signal-to-noise ratio for the optical waveform, resulting in a relatively accurate measurement.

Still other embodiments are within the scope of the following claims.

What is claimed is:

1. A monitor for measuring blood pressure from a patient comprising:
a housing comprising a first surface;
a first sensor comprised by the first surface, the first sensor comprising: i) an optical system comprising at least one light source that generates optical radiation, and a photodetector oriented to collect radiation after it irradiates the patient and in response generate an optical signal; and ii) a first electrode;
a second sensor comprising a second electrode paired with the first electrode and configured to collect an electrical signal from the patient; and
a microprocessor in electrical communication with the first and second sensor, the microprocessor configured to receive the optical and electrical signals and process these signals with an algorithm to determine systolic and diastolic blood pressure.

2. The monitor of claim 1, wherein both the first and second sensor are comprised by the first surface.

3. The monitor of claim 1, wherein the housing further comprises a second surface that comprises the second sensor.

4. The monitor of claim 3, wherein the first surface is a back surface of the monitor, and the second surface is a front surface of the monitor.

5. The monitoring device of claim 1, wherein the first surface comprises both the light-emitting diode and photodetector.

6. The monitoring device of claim 5, wherein the first sensor comprises a first electrode that surrounds both the light source and the photodetector.

7. The monitoring device of claim 1, wherein the first electrode is a metal pad.

8. The monitoring device of claim 1, wherein the first surface comprises a depression that comprises the first sensor.

9. The monitoring device of claim 8, wherein the depression comprises a width that is approximately the size of a human finger.

10. The monitoring device of claim 1, wherein the light source is a light-emitting diode.

11. The monitoring device of claim 1, wherein the light source is a laser diode.

12. The monitoring device of claim 1, further comprising at least two light sources.

13. The monitoring device of claim 12, wherein a first light source emits radiation in a red spectral region, and a second light source emits radiation in an infrared spectral region.

14. The monitoring device of claim 13, wherein the microprocessor further comprises an algorithm for calculating pulse oximetry.

15. The monitoring device of claim 1, wherein the microprocessor further comprises an algorithm for calculating heart rate.

16. The monitoring device of claim 1, further comprising a component that attaches to the first surface to partially cover the first sensor, the component comprising the first light source.

17. The monitoring device of claim 16, wherein the component is a flexible strap.

18. The monitoring device of claim 16, wherein the component is a plastic component comprised by the first surface.

19. A monitoring device for measuring blood pressure comprising:
a housing comprising first and second surfaces;
a first sensor comprised by the first surface, the first sensor comprising: i) an optical system comprising at least one light source and a photodetector configured to generate an optical waveform; and ii) a first electrode;
a second sensor comprised by the second surface, the second sensor comprising a second electrode paired with the first electrode to generate an electrical waveform; and
a microprocessor in electrical communication with the first and second sensor, the microprocessor configured to operate an algorithm that receives and processes the optical and electrical waveforms to measure systolic and diastolic blood pressure.

20. A monitoring device for measuring blood pressure comprising:
a housing comprising a first surface;
a first sensor comprised by the first surface, the first sensor comprising: i) an optical system comprising at least one light source and a photodetector configured to generate an optical waveform; and ii) a first electrode;
a second sensor comprised by the first surface, the second sensor comprising a second electrode paired with the first electrode to generate an electrical waveform;
a microprocessor in electrical communication with the first and second sensor, the microprocessor configured to operate an algorithm that receives and processes the optical and electrical waveforms to measure systolic and diastolic blood pressure values; and
a wireless transmitter comprising a transceiver for transmitting the systolic and diastolic blood pressure values to an external receiver.
